(19) United States
(12) Patent Application Publication
(10) Pub. No.: US 2017/0079696 A1
(43) Pub. Date: Mar. 23, 2017

(54) Spinal Fixation System

(71) Applicant: Blackstone Medical, Inc., Lewisville, TX (US)

(72) Inventors: Clinton Walker, Frisco, TX (US); John Lovell, Celina, TX (US)

(21) Appl. No.: 14/862,083

(22) Filed: Sep. 22, 2015

(51) Int. Cl. A61B 17/70

(52) U.S. Cl. CPC A61B 17/7088; A61B 2017/00526

(57) ABSTRACT

A spinal fixation system may comprise a body, a tower, a set screw holder, and a set screw. A body may comprise a saddle, at least two tabs connected to and extending proximally from the saddle, a receiving channel disposed between the at least two tabs, and a securing mechanism, such as internal threads. A tower may comprise a central shaft configured to be received into the receiving channel, a cannula, at least two panels, an enlarged section, and an attachment feature configured to slide proximally and distally between the at least two panels and the enlarged section. A set screw holder may comprise a receiver and an insert. A receiver may comprise a passageway and a set screw engaging feature. The insert may be is sized to be received into the passageway of the receiver and may be configured to engage the set screw engaging feature.

### FIELD OF THE DISCLOSURE

The present disclosure relates, in some embodiments, to spinal fixation systems. More specifically, embodiments of the present disclosure may provide for spinal fixation systems that allow for rod reduction, direct vertebral rotation, compression, distraction, counter-torque during tightening, and other surgical maneuvers.

### BACKGROUND OF THE DISCLOSURE

The spinal column of bones is a highly complex anatomical structure that includes over 20 bones coupled to one another, housing and protecting critical elements of the nervous system having innumerable peripheral nerves and circulatory bodies in close proximity. Despite its complexity, the spine is a highly flexible structure, capable of a high degree of curvature and twist in nearly every direction. The more than 20 discrete bones of an adult human spinal column are anatomically categorized as one of four classifications—cervical, thoracic, lumbar, or sacral—and are coupled together sequentially to one another by a tri-joint complex that consists of an anterior disc and two posterior facet joints. The anterior discs of adjacent bones are cushioned by cartilage spacers referred to as intervertebral discs or vertebrae. The cervical portion of the spine comprises the top of the spine up to the base of the skull and includes the first seven vertebrae. The intermediate 12 bones are thoracic vertebrae, and connect to the lower spine comprising the 5 lumbar vertebrae. The base of the spine comprises sacral bones, including the coccyx, which are fused together. With its complex nature, however, there is also an increased likelihood that surgery may be needed to correct one or more spinal pathologies.

Various systems exist for connecting fastener elements (e.g., pedicle screws) to bones for the purposes vertebral fixation. Such systems may use a plurality of bone screws fitted in saddles, wherein a plurality of saddles are aligned using a mounting rod. Alignment of mounting rods through positionally fixed saddles may be challenging. If a saddle is not in an appropriate position or an aligned position, the bone screw may have to be removed or repositioned, or the saddle may have to be refitted. In some situations, the mounting rod may have to be bent to align with the saddle at a fixed position. In some situations, additional components, such as offset connectors, may have be introduced to a bone screw system, such that a series of saddles may be aligned and secured by a mounting rod.

Further, in some situations, additional devices may be needed to allow for various adjustments or tightening of the implanted pedicle screws. Such adjustments may involve rod reduction, direct vertebral rotation, compression, distraction, counter-torque during tightening, and other surgical maneuvers. The need for additional devices or adjustments may, in some situations, frustrate or impair the desirability of minimally invasive surgeries.

### SUMMARY

Accordingly, a need has arisen for improved spinal fixation systems that may allow for operations such as rod reduction, direct vertebral rotation, compression, distraction, and counter-torque during tightening while maintaining the advantages of minimally invasive surgeries.

In some embodiments, the present disclosure provides for spinal fixation systems. A spinal fixation system may comprise a body, a tower, a retention device, and a securing device. As used herein, a retention device may be a set screw holder, and a securing device may be a set screw. One of ordinary skill in the art would appreciate other appropriate retention devices and other appropriate securing devices in accordance with the present disclosure. The body may have a proximal end and a distal end. The body may comprise a saddle at the distal end of the body; at least two tabs connected to and extending proximally from the saddle; a receiving channel disposed between the at least two tabs and extending into the saddle; and a securing mechanism. The tower may have a proximal end and a distal end. The tower may comprise a central shaft having a proximal end and a distal end, wherein the central shaft is configured to be received into at least a portion of the receiving channel of the body. The tower may further comprise a cannula disposed along a longitudinal axis of the central shaft, wherein the longitudinal axis extends from the proximal end to the distal end of the central shaft. The tower may further comprise at least two panels disposed at the distal end of the central shaft and extending distally from the central shaft; an enlarged section disposed at the proximal end of the central shaft; and an attachment feature disposed at the proximal end of the central shaft, wherein the attachment feature is configured to slide proximally and distally between the at least two panels and the enlarged section.

The set screw holder may comprise a receiver having a proximal end and a distal end, wherein the receiver may comprise a passageway extending from the proximal end to the distal end of the receiver. The receiver may comprise a securing device engaging feature disposed at the distal end of the receiver. As used herein, a securing device engaging feature may refer to a set screw engaging feature. One of ordinary skill in the art would appreciate other appropriate securing device engaging features in accordance with the present disclosure. The receiver may be sized to be received into the cannula of the tower. The set screw holder may further comprise an insert having a proximal end and a distal end. The insert may be sized to be received into the passageway of the receiver; wherein the distal end of the insert may be configured to engage the set screw engaging feature. A set screw may have a proximal end and a distal end. The set screw may comprise a securing feature at the proximal end of the set screw, wherein the securing feature may be configured to engage with the set screw engaging feature of the set screw holder.

In some embodiments, the securing mechanism of the body may comprise internal threads disposed along an internal section of a distal end of the at least two tabs and a proximal end of the saddle. The set screw may comprise external threads configured to mate with the securing mechanism of the body.

In some embodiments, the enlarged section may comprise an annular protrusion relative to the central shaft. An exterior of the attachment feature may comprise a geometry selected from one of a triangular, rectangular, pentagonal, hexagonal, heptagonal, or octagonal geometry. Each of the at least two panels may each comprise a plurality of recesses disposed on an exterior surface of the at least two panels. The spinal fixation system may further comprise a compression tool or a distraction tool configured to engage with the plurality of recesses. In some embodiments, a distal end of the attachment feature may further comprise a first set of undercuts configured to receive the at least two tabs of the body, and a second set of undercuts configured to receive the at least two panels of the tower. The cannula of the tower may be sized such that the set screw cannot pass therethrough. A length of the insert of the set screw holder may be greater than a length of the receiver of the set screw holder. The set screw engaging feature may be configured to expand or retract in response to contact from the distal end of the insert. The insert of the set screw holder may further comprise an exterior tab disposed on the proximal end of the insert, wherein the exterior tab may bias against an internal feature disposed on an interior surface of the receiver. The set screw may be secured against the set screw engaging feature of the set screw holder when the set screw engaging feature is in an expanded configuration in the securing feature of the set screw. The central shaft of the tower may further comprise a threading section at the distal end of the central shaft, wherein the threading section may comprise exterior threads disposed within the cannula of the tower, wherein the exterior threads are configured to engage with the internal threads of the body.

According to another aspect, embodiments of the present disclosure may provide for methods of assembling and/or using a spinal fixation system. Methods may comprise disposing a set screw holder within a tower, securing a set screw to the set screw engaging feature of the set screw holder, providing a body having a proximal end and a distal end, inserting an initial combined arrangement of the set screw holder, the tower, and the set screw through the receiving channel of the body; and securing the initial combined arrangement against the securing mechanism of the body.

Securing the initial combined arrangement against the securing mechanism of the body may comprise rotating the initial combined arrangement, whereby rotating the initial combined arrangement secures the external threads of the set screw against the internal threads of the body.

In some embodiments, methods may further comprise securing an instrument handle against an exterior of the attachment feature, wherein an exterior of the attachment feature comprises a geometry selected from one of a triangular, rectangular, pentagonal, hexagonal, heptagonal, or octagonal geometry. Methods may further comprise securing an instrument against one of a plurality of recesses disposed on an exterior surface of the at least two panels. Methods may further comprise receiving the at least two tabs of the body into a set of undercuts of the attachment feature. Methods may further comprise biasing an exterior tab disposed on the proximal end of the insert against an internal feature disposed on an interior surface of the receiver.

In some embodiments, methods may further comprise positioning the set screw in a secured position such that at least a portion of the set screw is disposed within the saddle of the body; and releasing the set screw from the set screw holder by retracting the set screw engaging feature from the securing feature of the set screw, wherein the set screw engaging feature is configured to expand or retract in response to contact from the distal end of the insert. Methods may further comprise removing a releasable combined arrangement of the set screw holder and the tower through the receiving channel of the body. Methods may further comprise separating the at least two tabs of the body from the saddle of the body.

### BRIEF DESCRIPTION OF THE DRAWINGS

Some embodiments of the disclosure may be understood by referring, in part, to the present disclosure and the accompanying drawings, wherein:

FIG. 1 depicts a perspective view of various components of a spinal fixation system according to some embodiments of the present disclosure;

FIG. 2A depicts a perspective view of various components of a set screw holder according to some embodiments of the present disclosure;

FIG. 2B depicts a perspective view of a proximal portion of an insert of a set screw holder according to some embodiments of the present disclosure;

FIG. 2C depicts a perspective view of one configuration a distal portion of a receiver of a set screw holder according to some embodiments of the present disclosure;

FIG. 2D depicts a perspective view of another configuration of a distal portion of a receiver of a set screw holder according to some embodiments of the present disclosure;

FIG. 3A depicts a perspective view of a tower according to some embodiments of the present disclosure;

FIG. 3B depicts another perspective view of a tower according to some embodiments of the present disclosure;

FIG. 3C depicts a side view of a tower according to some embodiments of the present disclosure;

FIG. 3D depicts a cross-sectional side view of a tower according to some embodiments of the present disclosure;

FIG. 3E depicts another cross-sectional side view of a tower according to some embodiments of the present disclosure;

FIG. 3F depicts a perspective view of a proximal section of a tower according to some embodiments of the present disclosure;

FIG. 4 depicts a perspective view of a body according to some embodiments of the present disclosure;

FIG. 5A depicts a perspective view of assembling a set screw holder according to some embodiments of the present disclosure;

FIG. 5B depicts a perspective view of assembling a set screw holder and a tower according to some embodiments of the present disclosure;

FIG. 5C depicts another perspective view of assembling a set screw holder and a tower according to some embodiments of the present disclosure;

FIG. 5D depicts a perspective view of assembling a set screw holder, a tower, and a set screw according to some embodiments of the present disclosure;

FIG. 5E depicts a perspective view of assembling a set screw holder, a tower, a set screw, and a body according to some embodiments of the present disclosure;

FIG. 5F depicts another perspective view of assembling a set screw holder, a tower, a set screw, and a body according to some embodiments of the present disclosure;

FIG. 5G depicts a close-up see-through view of an attachment feature according to some embodiments of the present disclosure;

FIG. 5H depicts a perspective view of disassembling a set screw holder from a spinal fixation system according to some embodiments of the present disclosure;

FIG. 5I depicts a perspective see-through view of components of a spinal fixation system according to some embodiments of the present disclosure; and

FIG. 5J depicts a cross-sectional perspective view of components of a spinal fixation system according to some embodiments of the present disclosure.

The thousands digit correspond to the figure in which the feature appears while the hundreds, tens and ones digits correspond to the particular feature indicated. Similar structures share matching hundreds, tens and ones digits.

### DETAILED DESCRIPTION

The present disclosure relates, in some embodiments, to spinal fixation systems. Such spinal fixations systems may be advantageous for use in spinal surgeries (e.g. posterior pedicle screw fixation surgery). More specifically, embodiments of the present disclosure may provide for spinal fixation systems that allow for rod reduction, direct vertebral rotation, compression, distraction, counter-torque during tightening, and other surgical maneuvers. The present disclosure may advantageously provide for a spinal fixation system that allows for such maneuvers with a low number of total components, while maintaining the benefits of a minimally invasive design or a minimally invasive approach. In some embodiments, the spinal fixation system may be considered an “all-in-one” instrument or system to facilitate the aforementioned surgical maneuvers.

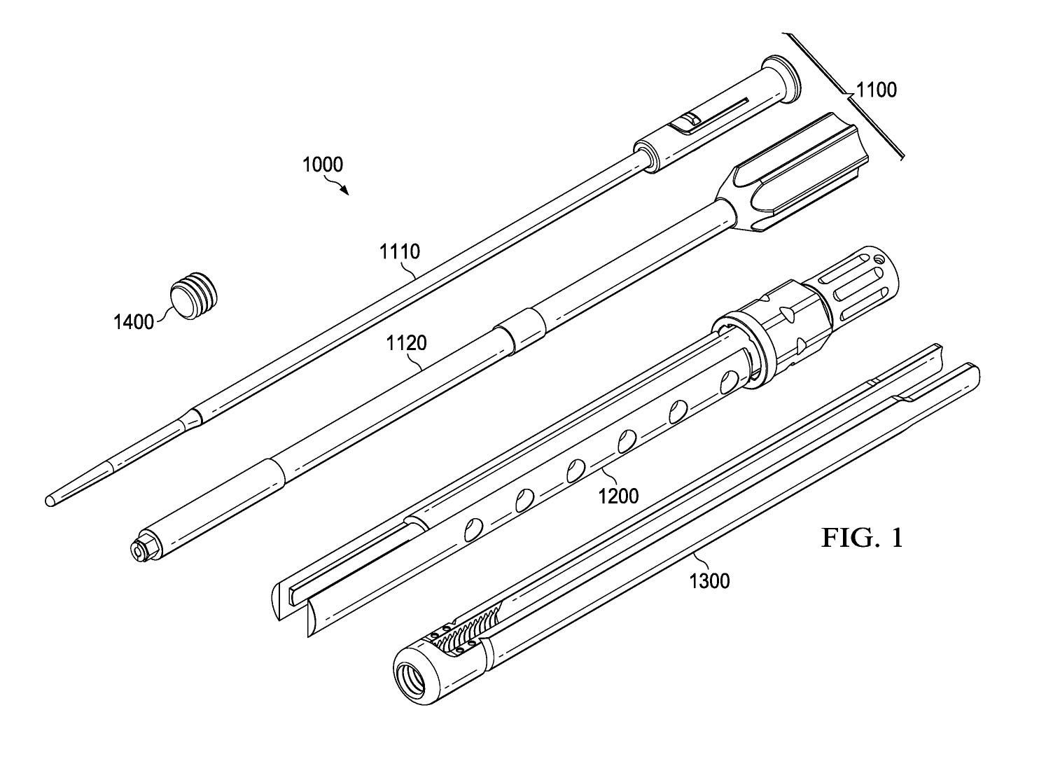

FIG. 1 depicts various components of a spinal fixation system 1000 according to an embodiment of the present disclosure. As depicted, the spinal fixation system 1000 may comprise a retention device 1100, a tower 1200, a body 1300, and a securing device 1400. As used herein, a retention device 1100 may be any mechanism that may hold a securing device 1400. A retention device 1100 may hold a securing device 1400 at one end and allow for the securing device 1400 to be otherwise transferred and/or secured within a body of a spinal fixation system. In some embodiments, a securing device 1400 may be a set screw. Consistently, in some embodiments, a retention device 1100 may be a set screw holder. Thus, the description provided herein may prefer to a retention device 1100 as a set screw holder 1100 and a securing device 1400 as a set screw 1400. However, such description is not intended to be limiting, and one of ordinary skill in the art would appreciate that securing devices 1400 other than a set screw 1400 may be appropriate, and that retention devices 1100 other than a set screw holder 1100 may be appropriate. As seen in FIG. 1, the set screw holder 1100 may comprise an insert 1110 and a receiver 1120. Usage of the various components as depicted in FIG. 1 may facilitate securing of the set screw 1400 into the body 1300 during a surgical setting. Various features of the set screw holder 1100, the tower 1200, and the body 1300 may also facilitate various surgical maneuvers such as rod reduction, direct vertebral rotation, compression, distraction, and counter-torque during tightening.

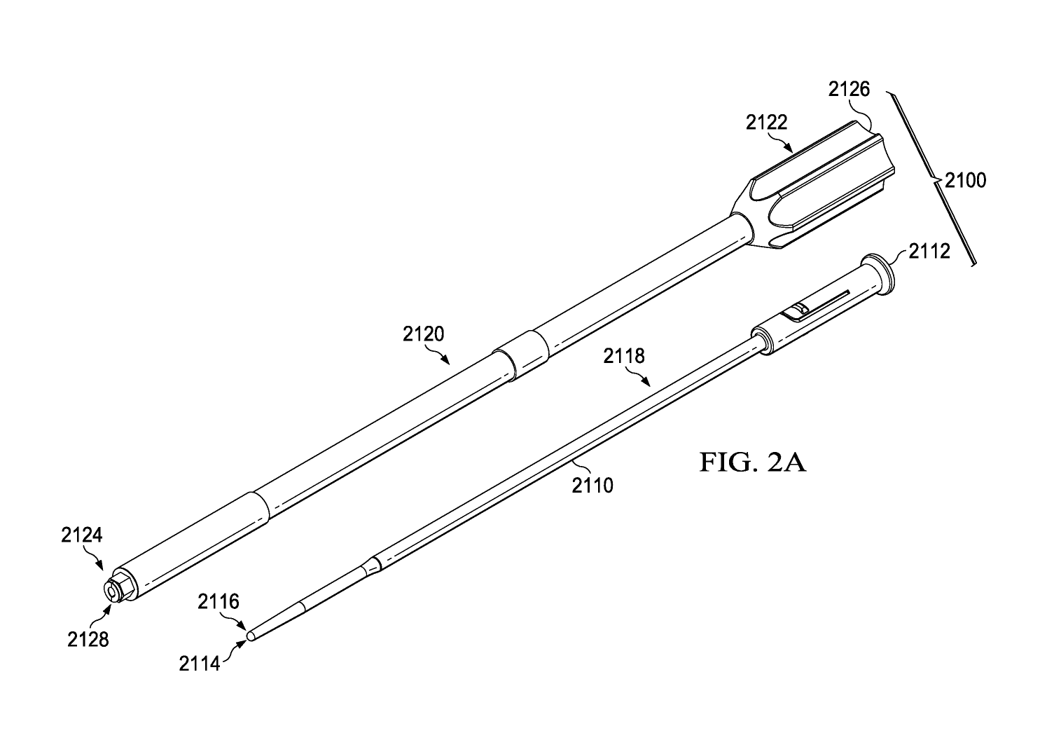

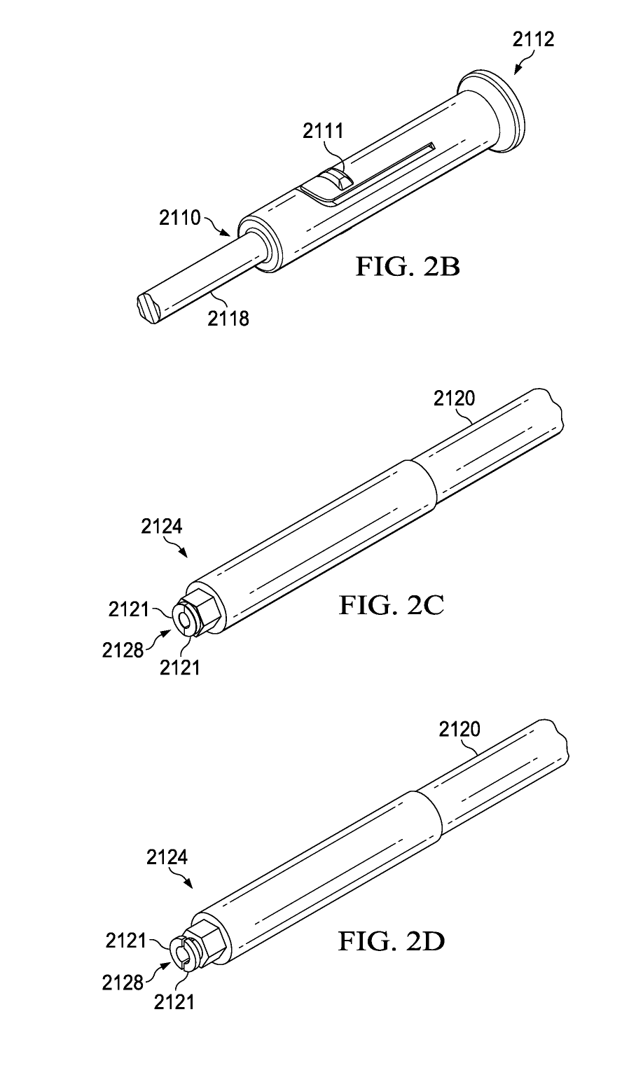

FIG. 2A depicts an example retention device 2100, such as a set screw holder 2100, according to an embodiment of the present disclosure. The set screw holder 2100 may function to hold or otherwise secure a set screw as it is being lowered into and secured against a body of a spinal fixation system. As depicted, the set screw holder 2100 may comprise an insert 2110 and a receiver 2120.

The receiver 2120 may have a proximal end 2122 and a distal end 2124. The proximal end 2122 may be larger or thicker than the rest of the receiver 2120. The larger proximal end 2122 may allow for easier handling or manipulation during a surgical operation. The larger proximal end 2122 may also prevent the entire receiver 2120 from being lowered into a tower of a spinal fixation system during a surgical operation.

The receiver 2120 may also comprise a passageway 2126. The passageway 2126 may extend from the proximal end 2122 to the distal end 2124 of the receiver 2120. In such manner, the receiver 2120 may comprise a substantially cylindrical geometry with a hollow center. The passageway 2126 may be sized so as to allow the insert 2110 to be received therein. Thus, the insert 2110 may slide into the passageway 2126 of the receiver 2120.

The receiver 2120 may further comprise a securing device engaging feature 2128 disposed at the distal end 2124. As used herein, a securing device engaging feature 2128 may be any feature or mechanism suitable to mate or engage with a securing device, such as a set screw. In some embodiments, a securing device may be a set screw. Thus, a securing device engaging feature 2128 as used herein may be refereed to as a set screw engaging feature 2128. Such description is not intended to be limiting, and one of ordinary skill in the art would appreciate that other securing device engaging features 2128 other than a set screw engaging feature 2128 may be suitable for use in accordance with the present disclosure. In some embodiments, the set screw engaging feature 2128 may be a feature that can be secured to or lock against a set screw of a spinal fixation system. In some embodiments, the set screw engaging feature 2128 may expand or retract to either lock or release a set screw. In such manner, the set screw engaging feature 2128 may allow the set screw holder 2100 to secure a set screw so that it can be lowered into a body of a spinal fixation system. Then, when a set screw is in a desirable position, the set screw engaging feature 2128 may release the set screw at the secured, desirable position in a body of a spinal fixation system.

The insert 2110 may be a component with a substantially elongated and cylindrical structure. The insert 2110 may comprise a proximal end 2112 and a distal end 2114. The insert 2110 may also comprise a main segment 2118. The main segment 2118 may be substantially cylindrical and may be sized to be received into the passageway 2126 of the receiver 2120.

The proximal end 2112 may be larger or thicker than the rest of the insert 2110. The larger proximal end 2112 may allow for easier handling or manipulation during a surgical operation. The larger proximal end 2112 may also prevent the entire insert 2110 from being lowered into the passageway 2126 of the receiver 2120 of a spinal fixation system during a surgical operation.

The distal end 2114 of the insert 2110 may be configured to engage the set screw engaging feature 2128 of the receiver 2120. In some embodiments, the distal end 2114 may comprise a pointed feature 2116 to promote interaction with the set screw engaging feature 2128. For example, the insert 2110 may be inserted into the passageway 2126 of the receiver 2120. The distal end 2114 of the insert 2110 may thus move through the passageway 2126 and be positioned at the distal end 2124 of the receiver 2120. The distal end 2114 of the insert 2110 may then interact with or exert pressure against an interior portion of the set screw engaging feature 2128. Said interaction or exertion of pressure may cause the set screw engaging feature 2128 to expand or retract. Thus, the insert 2110 may be used to either secure or release a set screw at the distal end 2124 of the receiver 2120. The set screw engaging feature 2128 may thus expand or retract in response to contact with the distal end 2114 of the insert 2110. The description of the distal end 2114 of the insert 2110 as the pointed feature 2116 is provided by way of example only. Other geometries or designs may be used to allow for interaction of the distal end 2114 within the set screw engaging feature 2128 of the receiver 2120.

In some embodiments, a length of the insert 2110 of the set screw holder 2100 may be greater than a length of the receiver 2120 of the set screw holder 2100. Such a design may allow for the proximal end 2112 of the insert 2110 to remain exposed when the insert 2110 is inserted into the passageway 2126 of the receiver 2120. Thus, the proximal end 2112 of the insert 2110 may still be manipulated when the insert 2110 is inserted into the passageway 2126 of the receiver 2120.

FIG. 2B depicts a close-up view of the proximal end 2112 of the insert 2110. As depicted, the proximal end 2112 may further comprise an exterior tab 2111. In some embodiments, the exterior tab 2111 may be a slight protrusion that may be used to secure or bias against an internal feature disposed on an interior surface of the receiver. The internal feature may be disposed within the passageway 2126 of the receiver 2120. Interaction of the internal feature of the receiver 2120 and the exterior tab 2111 of the insert may allow the insert 2110 to be at least partially secured in a particular position when inserted into the passageway 2126. Thus, the exterior tab 2111 and the internal feature promotes added stability during the assembly and usage of a spinal fixation system of the present disclosure.

FIG. 2C and FIG. 2D depict close-up views of the distal end 2124 of the receiver 2120. As depicted, the set screw engaging feature 2128 may be disposed at the distal end 2124 of the receiver 2120. The set screw engage feature 2128 may comprise two segments 2121, wherein each segment 2121 has a substantially semi-circular geometry. FIG. 2C depicts the set screw engaging feature 2128 in a retracted position or state. In a retracted position or state, the segments 2121 are positioned relatively close to one another. The close positioning of the segments 2121 may allow a set screw to slide onto the set screw engaging feature 2128.

In some embodiments, the distal end 2114 of the insert 2110 may be received into and slide through the passageway 2126 and be positioned at the distal end 2124 of the receiver 2120. In this position, the distal end 2114 of the insert 2110 may exert pressure or interact with the set screw engaging feature 2128. Exerting pressure or interacting with the set screw engaging feature 2128 may cause the set screw engaging feature 2128 to move into an expanded position or state. FIG. 2D depicts the set screw engaging feature 2128 in an expanded position or state. In an expanded position or state, the segments 2121 may be further apart from each other. The further positioning of the segments 2121 may allow a set screw to be secured against the segments 2121 of the set screw engaging feature 2128. The design and geometries of the set screw engaging feature 2128 as depicted herein is provided by way of example only. Other designs and geometries may be used to allow for a set screw to be secured against the set screw engaging feature 2128 of the set screw holder 2100.

Dimensions of the set screw holder 2100 may be varied to accomplish various functional objectives. In some embodiments, the insert 2110 of the set screw holder 2100 may be about 150 mm to about 210 mm in length. A center portion of the insert 2110 may have a diameter of about 3 mm to about 5 mm. A proximal end 2112 of the insert 2110 may have a diameter of about 6 mm to about 9.5 mm. A distal end of 2114 of the insert 2110 may have a diameter of about 1 mm to about 2.5 mm.

In some embodiments, the receiver 2120 of the set screw holder 2100 may be about 140 mm to about 190 mm in length. A center portion of the receiver 2120 may have a diameter of about 6 mm to about 7 mm. A proximal end 2122 of the receiver 2120 may have a diameter of about 10.5 mm to about 12.5 mm. The set screw engaging feature 2128 of the receiver 2120 may have a diameter of about 4 mm to about 5 mm in a retracted state. In an expanded state, the set screw engaging feature 2128 may have a diameter of about 5 mm to about 6 mm. In an expanded state, segments 2121 of the set screw engaging feature 2128 may be spaced apart by about 1 mm. The passageway 2126 may have a diameter of about 3.5 mm to about 6.5 mm.

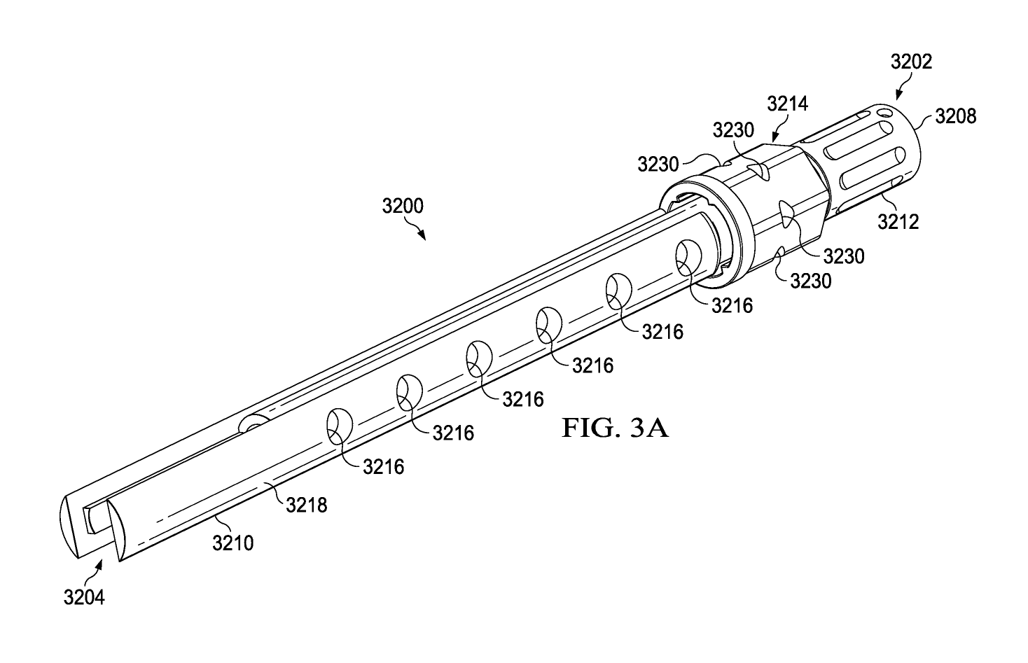

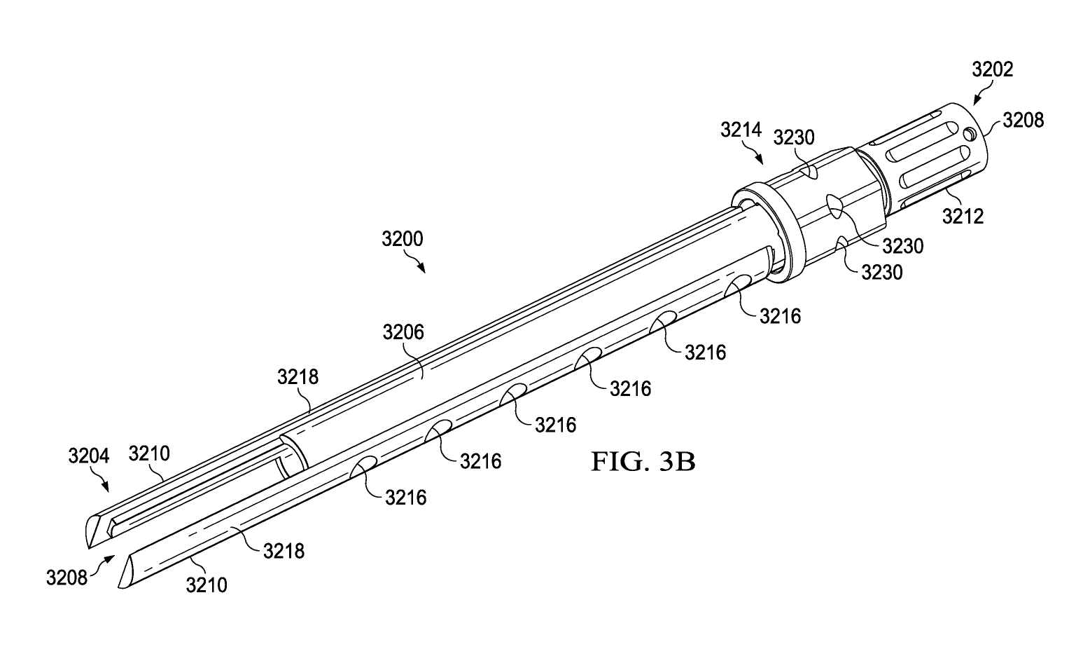

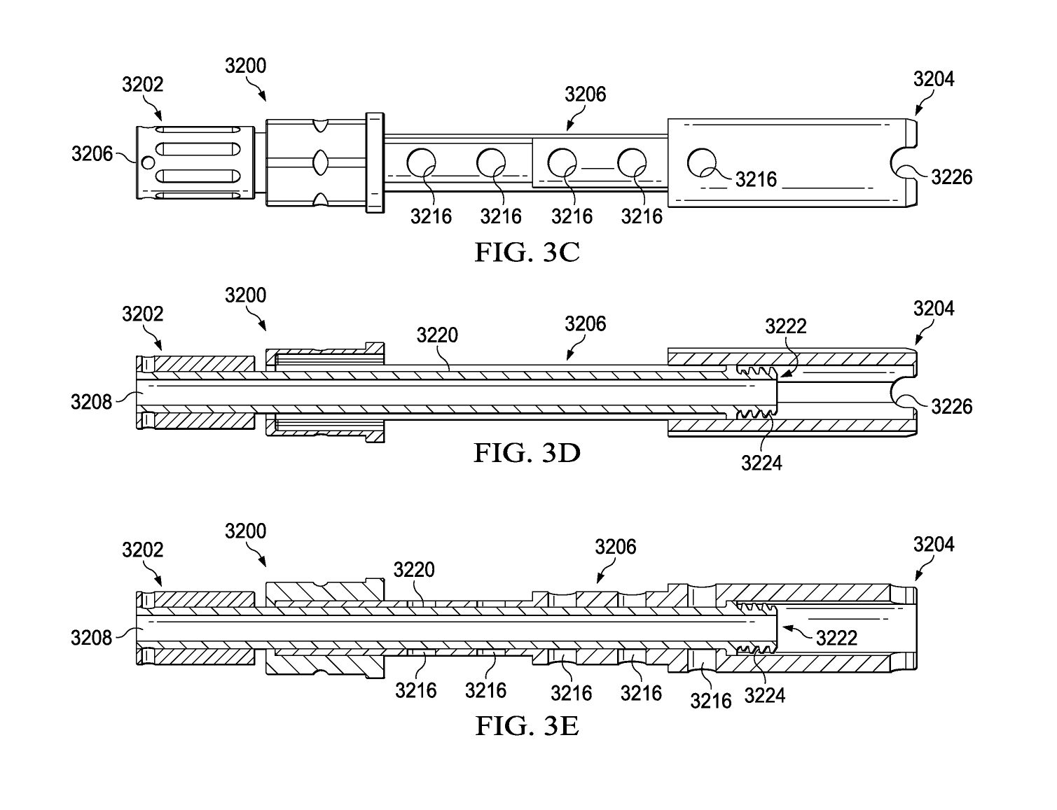

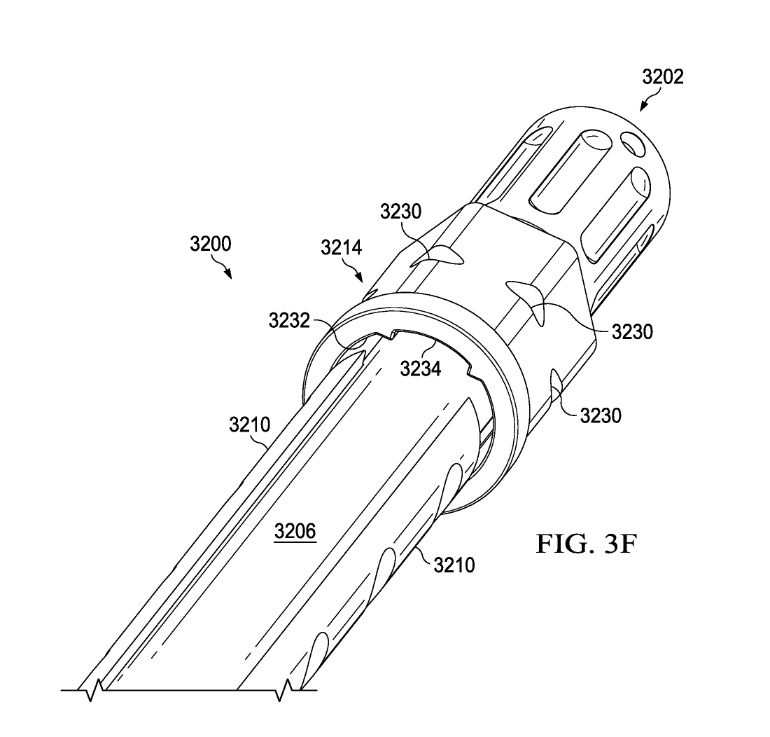

FIG. 3A and FIG. 3B depict an example tower 3200 according to some embodiments of the present disclosure. As depicted, the tower 3200 may have a proximal end 3202 and a distal end 3204. The tower 3200 may also comprise a central shaft 3206. The central shaft 3206 may comprise a substantially cylindrical geometry and may be sized to fit at least partially within the channel of a body of a spinal fixation system.

Further, the tower 3200 may comprise a cannula 3208. The cannula 3208 may be a hollow center of the tower 3200. The cannula 3208 may be disposed along a longitudinal axis of the central shaft 3206, wherein the longitudinal axis extends from the proximal end 3202 of the tower 3200 to the distal end 3204 of the tower 3200. The cannula 3208 may be sized to receive a set screw holder of a spinal fixation system. Further, the cannula 3208 may be sized such that a set screw cannot fit or slide through the cannula 3208. As a result, a set crew may not pass through the cannula 3208. In some embodiments, a set screw holder is longer than the tower 3200. When a set screw holder is received into the tower 3200, a proximal end of the set screw holder may still be exposed and may thus be readily manipulated and adjusted. Further, when the set screw holder is received into the tower 3200, a distal end of the set screw holder may extend distally from the distal end 3204 of the tower 3200. Thus, the set screw holder may engage a set screw and secure said set screw when inserted in the central shaft 3206 of the tower 3200.

The proximal end 3202 of the tower 3200 may serve as a handle from which to hold and/or position the tower 3200. The cannula 3208 of the tower 3200 may provide for an opening at the proximal end 3202 of the tower 3200. Thus, a set screw holder may be inserted into the cannula 3208 at the proximal end 3202. The proximal end 3202 of the tower 3200 may comprise an enlarged section 3212. The enlarged section 3212 may be disposed at a proximal end of the central shaft 3206 or may be disposed proximal to the central shaft 3206. In some embodiments, the enlarged section 3212 may comprise an annular protrusion relative to the central shaft 3206. The enlarged section 3212 may have a greater diameter than the central shaft 3206. The description of the enlarged section 3212 as an annular protrusion or having a greater diameter is provided by way of example only. Other designs or an enlarged section 3212 may also be suitable. For example, any protrusion from the central shaft 3206 of the enlarged section 3212 may not be completely circumferential. Further, the enlarged section 3212 may not be circular in geometry. In some embodiments, the enlarged section 3212 may comprise a polygonal geometry such a hexagon or a square.

The tower 3200 may further comprise at least two panels 3210 disposed along the sides of the central shaft 3206. In some embodiments, the tower 3200 may comprise two panels 3210 disposed on opposing sides of the central shaft 3206 of the tower 3200. The panels 3210 may be disposed at distal ends of the central shaft 3206 and may extend distally from the central shaft 3206 so that they extend beyond the distal end of the central shaft 3206.

The tower 3200 may further comprise a plurality of recesses 3216 disposed on an exterior surface 3218 of the at least two panels 3210. In some embodiments, the plurality of recesses 3216 may be a series of holes. The plurality of recesses 3216 may be equally sized and equally spaced along the exterior surface 3218 of the at least two panels 3210. The plurality of recesses 3216 may allow an instrument to engage with the tower 3200 to adjust various components of a spinal fixation system. For example, an instrument may be used to engage with one of the plurality of recesses 3216 to rotate the tower 3200. In some embodiments, such instruments may be a compressor or distractor instrument. The plurality of recesses 3216 may be various designs or geometries. Further, the plurality of recesses 3216 may differ in design, geometry, and/or size from one another. In some embodiments, the plurality of recesses 3216 are circular. In some embodiments, there may be five recesses 3216 disposed on the exterior surface 3218 of each panel 3210. In some embodiments, there may be six recesses 3216 disposed on the exterior surface 3218 of each panel 3210.

FIG. 3C, FIG. 3D, and FIG. 3E depict various views of a tower 3200 according to some embodiments of the present disclosure. As depicts, the tower 3200 may comprise a central shaft 3206, a proximal end 3202, and a distal end 3204. A plurality of recesses 3216 may be disposed along the central shaft 3206. In some embodiments, the central shaft 3206 of the tower 3200 may further comprise an internal shaft 3220. The internal shaft 3220 may span substantially the length of the central shaft 3206. The internal shaft 3220 may function to provide additional features internal to the central shaft 3206 of the tower 3200. Further, the internal shaft 3220 may define the diameter of the cannula 3208, and may be sized to receive a set screw holder of a spinal fixation system.

The internal shaft 3220 may comprise a threading section 3222. The threading section 3222 may comprise a series of external threads 3224 within the interior of the central shaft 3206 of the tower 3200. The external threads 3224 may thus be disposed within the cannula 3208 of the tower 3200. In some embodiments, internal shaft 3220 may rotate within the tower 3220 such that the external threads 3224 may freely rotate to engage with corresponding threading structures. The external threads 3224 may be designed to engage or mate with corresponding internal threads of a body of a spinal fixation system. In some embodiments, when a set screw holder is received within the cannula 3208 of the tower 3200, a set screw may then be secured to a distal end of the set screw holder. External threads of a set screw may then align with the external threads 3224 of the internal shaft 3220 of the tower 3200. Thus, in some embodiments, when a combined structure of the tower 3200, a set screw holder, and a set screw is secured into a body, both the external threads of the set screw and the external threads 3224 may be secured to the body. Such use of external threads 3224 may advantageously provide for greater stability of a spinal fixation system during surgical maneuvers.

In some embodiments, a distal end 3204 of the tower 3200 may comprise curved features 3226. The curved feature 3226 may be disposed or positioned so that it can bias against a corresponding feature. For example, when the tower 3200 is inserted into a body, the curved features 3226 may be used to exert biasing force or pressure against a mounting rod, which may have a corresponding curvature. The curved features 3226 may thus advantageously improve stability of the spinal fixation system and may increase the amount of compressive forces that may be exerted on components within a saddle of a body of a spinal fixation system. Further, the curved feature 3226 may also advantageously provide clearance from a corresponding feature.

Referring back to FIG. 3A and FIG. 3B, the tower 3200 may further comprise an attachment feature 3214. The attachment feature 3214 may also be disposed at the proximal end 3202 of the tower 3200. However, the attachment feature 3214 may be disposed at a lower position or distally from the enlarged section 3212. The attachment feature 3214 may be loosely secured or slidably secured to the tower 3200. The attachment feature 3214 may be configured to slide proximally and distally between the at least two panels 3210 and the enlarged section 3212. In such manner, the attachment feature 3214 may slide longitudinally up and/or down along the central shaft 3206 of the tower 3200. Movement of the attachment feature 3214 along a longitudinal axis may be limited by the panels 3210, the enlarged section 3212, and a body, if present. In some embodiments, the attachment feature 3214 may slide distally so that a portion of the panels 3210 or a body is covered and therefore secured within the attachment feature 3214. Further, the attachment feature 3214 may also have rotational movement around the central shaft 3206. In some embodiments, the attachment feature 3214 may serve as a support ring to securing other components of a spinal fixation system.

In some embodiments, the attachment feature 3214 may comprise a particular geometry. Such geometries may include a triangular, rectangular, pentagonal, hexagonal, heptagonal, or octagonal geometry. For example, the attachment feature 3214 may comprise an exterior that may be a hexagonal geometry. Such a particular geometry may allow a corresponding geometry to fit or be secured against the exterior of the attachment feature 3214. For example, a counter-torque wrench having an instrument handle with a corresponding hexagonal recess may fit over the attachment feature 3214 having a hexagonal geometry. In such manner, the attachment feature 3214 may provide a point of interaction whereby additional components or tools may be used to perform actions such as rod reduction, direct vertebral rotation, compression, distraction, counter-torque during tightening, and other surgical maneuvers. The attachment feature 3214 may also comprise a plurality of notches 3230 along an exterior surface. In some embodiments, the notches 3230 may be small grooves disposed laterally along the exterior of the attachment feature 3214. The notches 3230 may provide added frictional support so as to better secure another component or tool to the attachment feature 3214.

The description of the attachment feature 3214 as comprising a hexagonal geometry is provided by way of example only. Other designs or geometries may be appropriate to allow another component or tool to be secured to the attachment feature 3214. For example, other appropriate geometries may include, but is not limited to, circular, ovoid, triangular, rectangular, pentagonal, and octagonal.

FIG. 3F depicts a perspective view of the tower 3200. As depicted, the attachment feature 3214 may be disposed relatively high on the tower 3200, or relatively close to the proximal end 3202 of the tower 3200. As previously described, the attachment feature 3214 may comprise a plurality of notches 3230 disposed laterally across the attachment feature 3214. The attachment feature 3214 may further comprise at least one set of undercuts along an underside of the attachment feature 3214. In some embodiments, the attachment feature 3214 may comprise a first set of undercuts 3234 configured to receive correspondingly sized tabs from a body of a spinal fixation system. When the tower 3200 is secured to a body, tabs of the body may be received into and secured within the first set of undercuts 3234 of the attachment feature 3214 of the tower 3200. The attachment feature 3214 may further comprise a second set of undercuts 3232. The second set of undercuts 3232 may be sized to receive the panels 3210 of the tower 3200. Accordingly, in some embodiments, the attachment feature 3214 may slide distally and, if the second set of undercuts 3232 is aligned with the panels 3210, at least a proximal portion of the panels 3210 may be received into and secured within the second set of undercuts 3234.

In some embodiments, the attachment feature 3214 comprises two first undercuts 3234 and two second undercuts 3232. The two pairs of undercuts may be positioned perpendicular from each other relative to the central shaft 3206. Thus, when the tower 3200 is secured to a body, the attachment feature 3214 may be distally lowered so that tabs of the body are at least partially received into the first set of undercuts 3234 and panels 3210 of the tower 3200 are at least partially received into the second set of undercuts 3234.

Dimensions of the tower 3200 may be varied to accomplish various functional objectives. In some embodiments, the tower 3200 may have a length of about 110 mm to about 165 mm. The center shaft 3206 may have a length of about 80 mm to about 140 mm. Further, the center shaft 3206 may have a diameter of about 10 mm to about 11 mm. Each of the plurality of panels 3210 may have a length of about 60 mm to about 120 mm. Each of the plurality of recesses 3216 may have a diameter of about 3 mm to about 5.5 mm. In some embodiments, each of the plurality of recesses 3216 may be spaced about by about 10 mm to about 25 mm. The cannula 3208 of the tower 3200 may have a diameter of about 7 mm to about 9 mm and a length of about 80 mm to about 165 mm. In some embodiments, the enlarged section 3212 may have a diameter of about 13 mm to about 15 mm and a length of about 10 mm to about 50 mm. The attachment feature 3214 may have a diameter of about 14 mm to about 25 mm and a length of about 10 mm to about 65 mm.

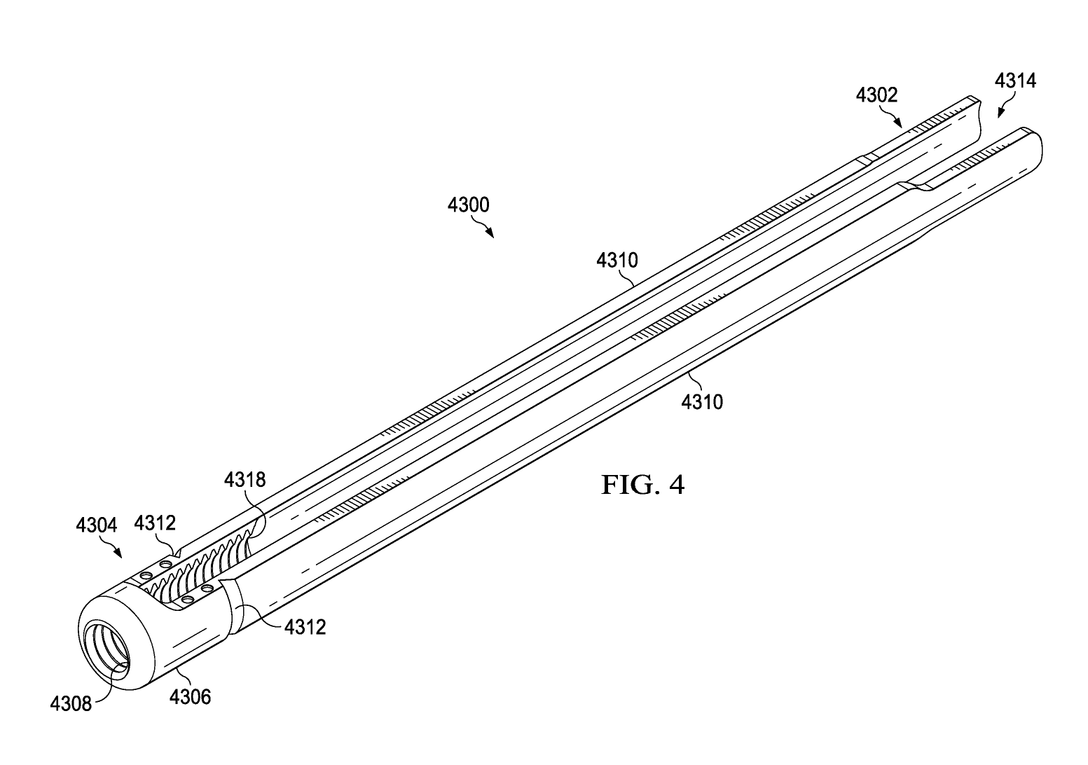

FIG. 4 depicts an example body 4300 according to some embodiments of the present disclosure. As depicted, the body 4300 may have a proximal end 4302 and a distal end 4304. The body 4300 may comprise a saddle 4306 at the distal end 4304 of the body 4300. The saddle 4306 may comprise a U-shaped profile. The saddle 4306 may comprise an opening 4308 at the distal end 4304 of the body 4300. In some embodiments, a bone screw may be disposed in the saddle 4306 so that a head of the bone screw rests within the saddle 4306 and a threading section of the bone screw extends through the opening 4308 of the body 4300.

The body 4300 may further comprise at least two tabs 4310 connected to and extending proximally from the saddle 4306. The at least two tabs 4310 may be spaced apart by a receiving channel 4314. The receiving channel 4314 of the body 4300 may be disposed between the at least two tabs 4310 and may extend into the saddle 4306. The receiving channel 4314 may connect to the opening 4308. Thus, various components may be lowered into the saddle 4306 or the opening 4308 through the receiving channel 4314. Examples of such components may include, but are not limited to, a bone screw, a mounting rod, and a pressure cap. For example, a bone screw may be lowered through the receiving channel 4314 such that a head of the bone screw rests within the saddle 4306 and a threading section of the bone screw extends distally through the opening 4308 of the body 4300. As another example, a mounting rod may be lowered in a lateral position down through the receiving channel 4314 of the body 4300. In some embodiments, a mounting rod may be lowered laterally so that various portions of the mounting rod are receiving through different receiving channels 4314 of different bodies 4300 of different spinal fixation systems that may be, for example, affixed to a pedicle region of a patient's spine. In such manner, the mounting rod may advantageously promote the alignment and positioning of the spinal fixation systems relative to one another.

The at least two tabs 4310 of the body 4300 may be connected to the saddle 4306 at respective indentations 4312. The indentations may allow the tabs 4310 to be more easily snapped off or otherwise removed from the saddle 4306. The tabs 4310 may be removed using particular instruments to avoid any unintended shrapnel from breaking from the body 4300 during removal or separation of the tabs 4310 from the saddle 4306.

In some embodiments, the receiving channel 4314 may be sized to receive a tower of a spinal fixation system. When a tower is inserted and disposed within the receiving channel 4314, the at least two tabs 4310 of the body 4300 may be positioned adjacent to or may flank the tower. The at least two tabs 4310 of the body 4300 may help to provide positionally security of a tower relative to the saddle 4306 of the body 4300. Further, a proximal section of the at least two tabs 4310 may be received into an undercut of an attachment section of the tower. In some embodiments, the at least two tabs 4310 may comprise a narrower width at a proximal section. A narrower width of a proximal section of the at least two tabs 4310 may advantageously allow the at least two tabs 4310 to be secured into an undercut of an attachment section of the tower.

The body 4300 may further comprise a securing mechanism. In some embodiments, a securing mechanism on the body 4300 may be internal threads 4318 disposed along an internal section of a distal end of the at least two tabs 4310. Each interior side of the at least two tabs 4310 may comprise internal threads 4318. The internal threads 4318 may span a distal end of the at least two tabs 4310 and a proximal end of the saddle 4306. The internal threads 4318 may be configured to mate with and secure external threads of a set screw. Accordingly, a set screw may be lowered through the receiving channel 4314. The set screw may then engage and be secured against the internal threads 4318 such that the set screw is disposed securely at a proximal portion of the saddle 4306. In some embodiments, other mechanisms may be used to allow a set screw to be secured therein. For example, a ratcheting locking system may be used in conjunction with a set screw comprising a cap with teeth to provide locking security against a spinal fixation system.

In some embodiments, the body 4300 may further comprise a ring structure at the proximal end 4302. Such ring structure may provide additional stability for some spinal fixation systems. However, such ring structure may also impede the insertion of a tower. Thus, in some embodiments, it may be advantageous to remove such ring structure before inserting a tower into the receiving channel 4314 of the body 4300.

Dimensions of the body 4300 may be varied to accomplish various functional objectives. In some embodiments, the body 4300 may have a length of about 90 mm to about 160 mm. In some embodiments, the saddle 4306 may have a height of about 10 mm to about 20 mm. Each of the at least two tabs 4310 may have a length of about 60 mm to about 150 mm. Each of the at least two tabs 4310 may have a width of about 6 mm to about 10 mm. In some embodiments, a proximal end of the at least two tabs 4310 may have a narrower width of about 4 mm to about 8 mm. The internal threads 4318 of the body 4300 may span about 6 mm to about 40 mm.

According to other aspects, the present disclosure may provide for methods of assembling and using a spinal fixation system. In some embodiments, methods may comprise preparing all instruments by removing all instruments from an instrument case and placing them in a disassembled state in preparation for use.

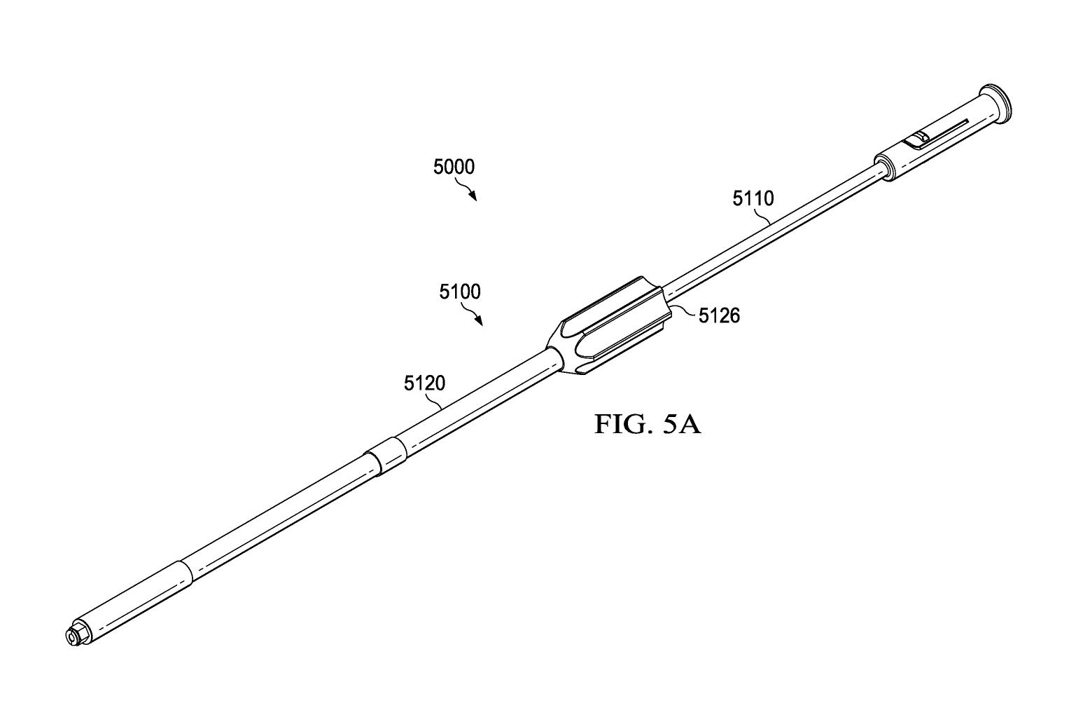

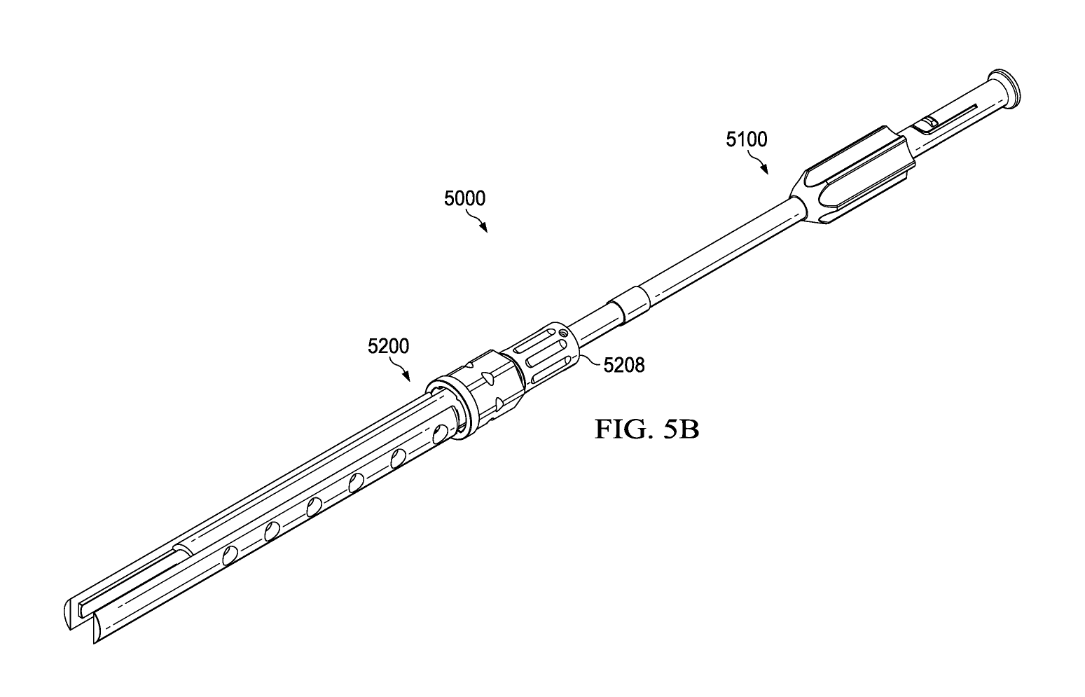

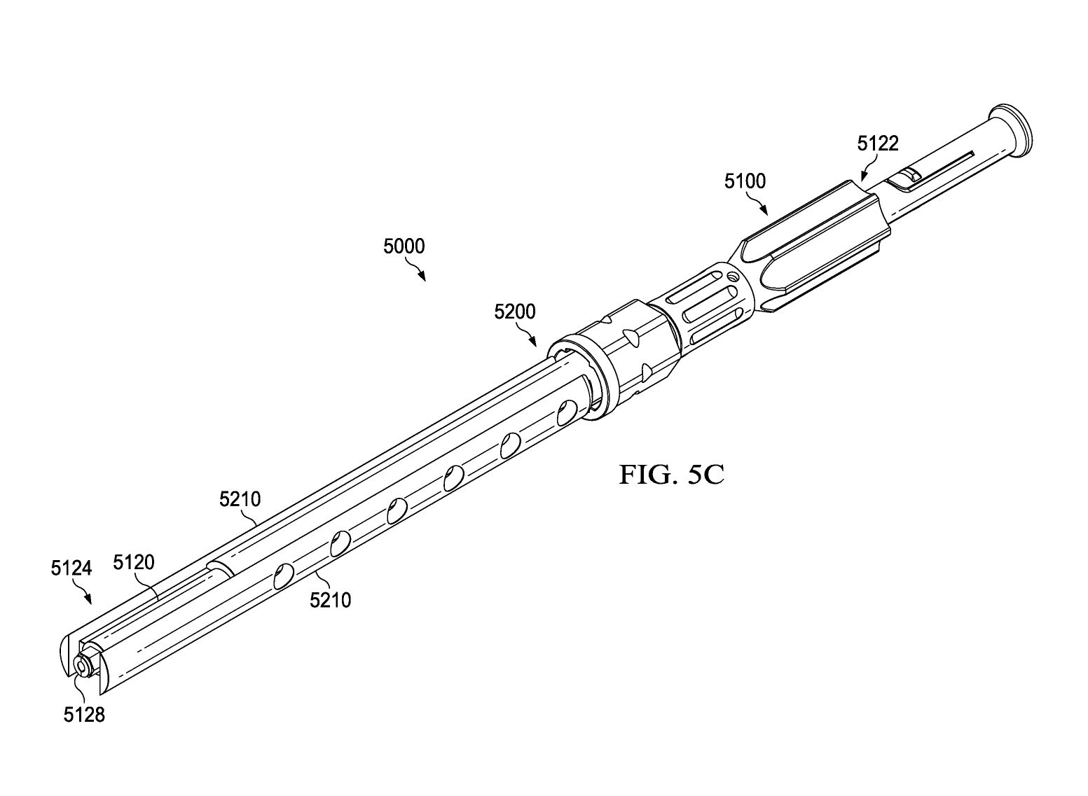

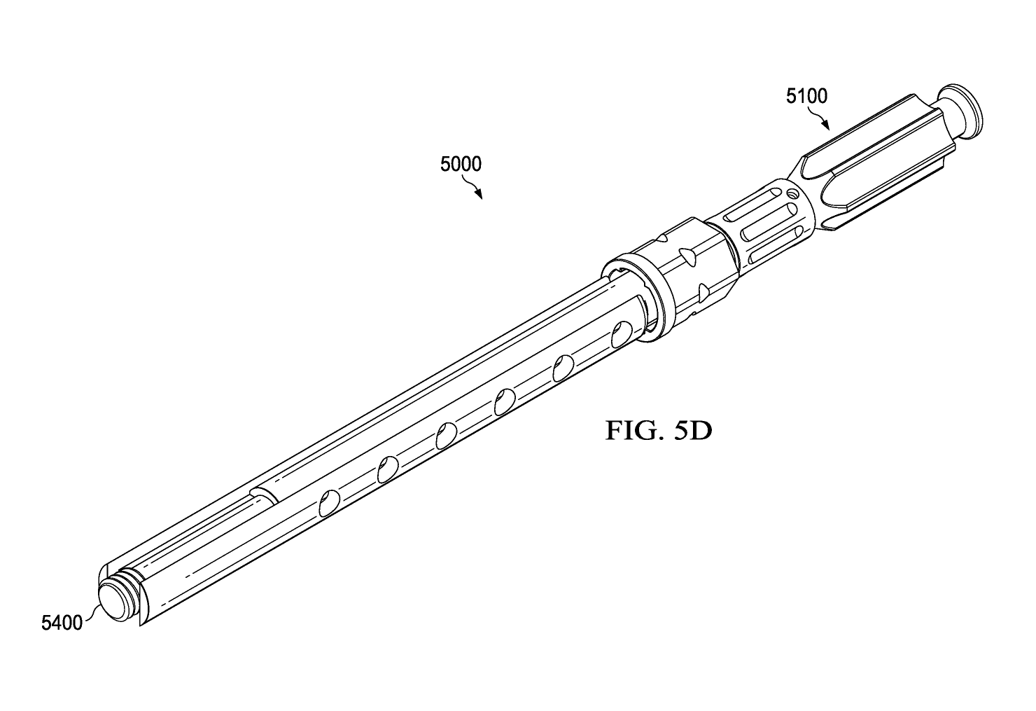

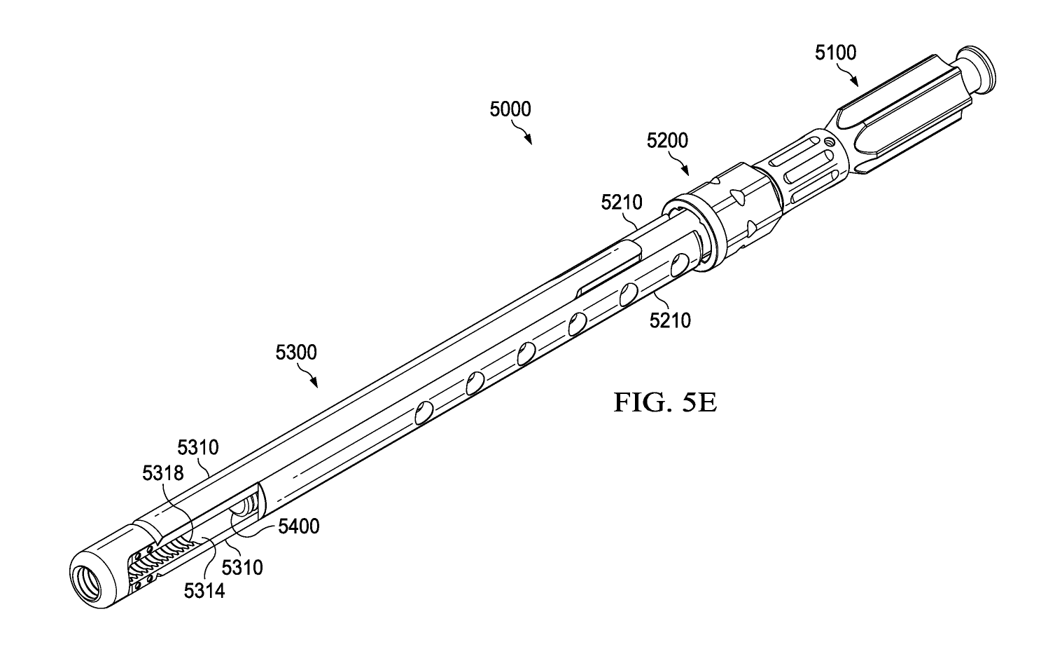

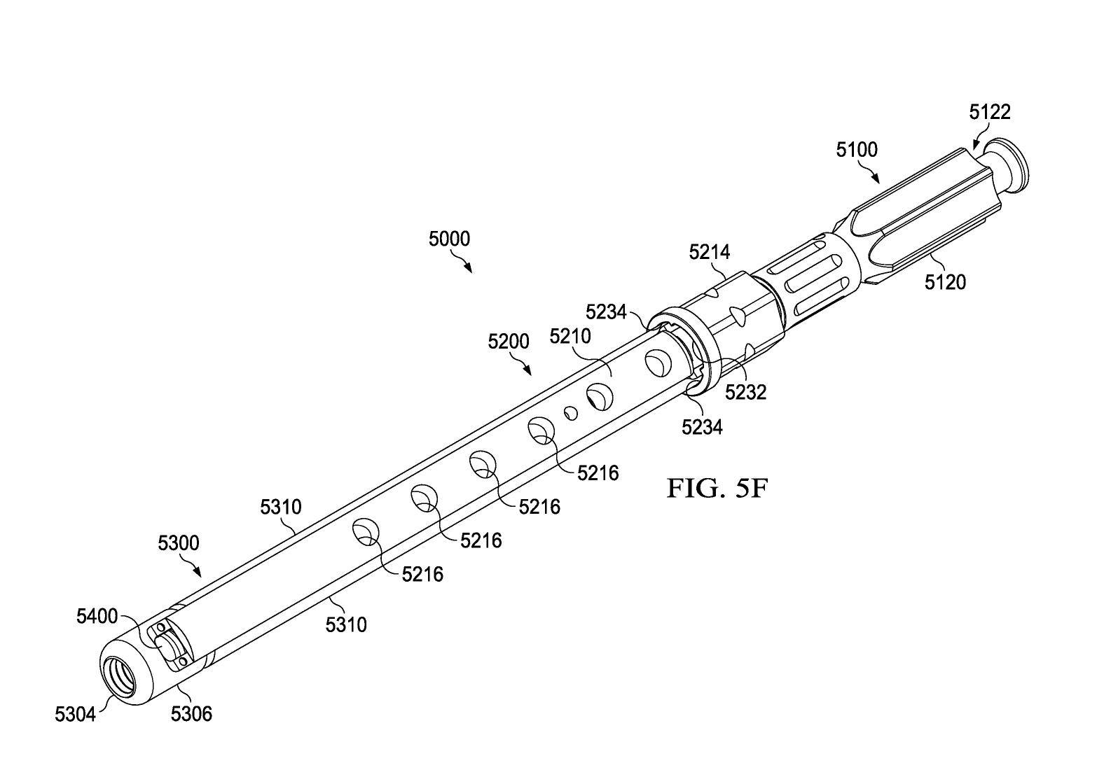

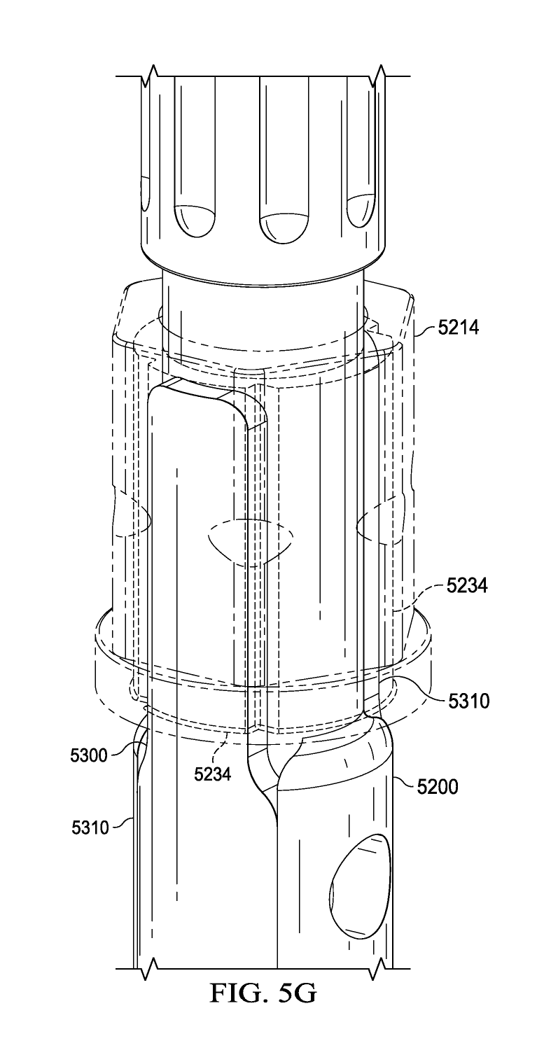

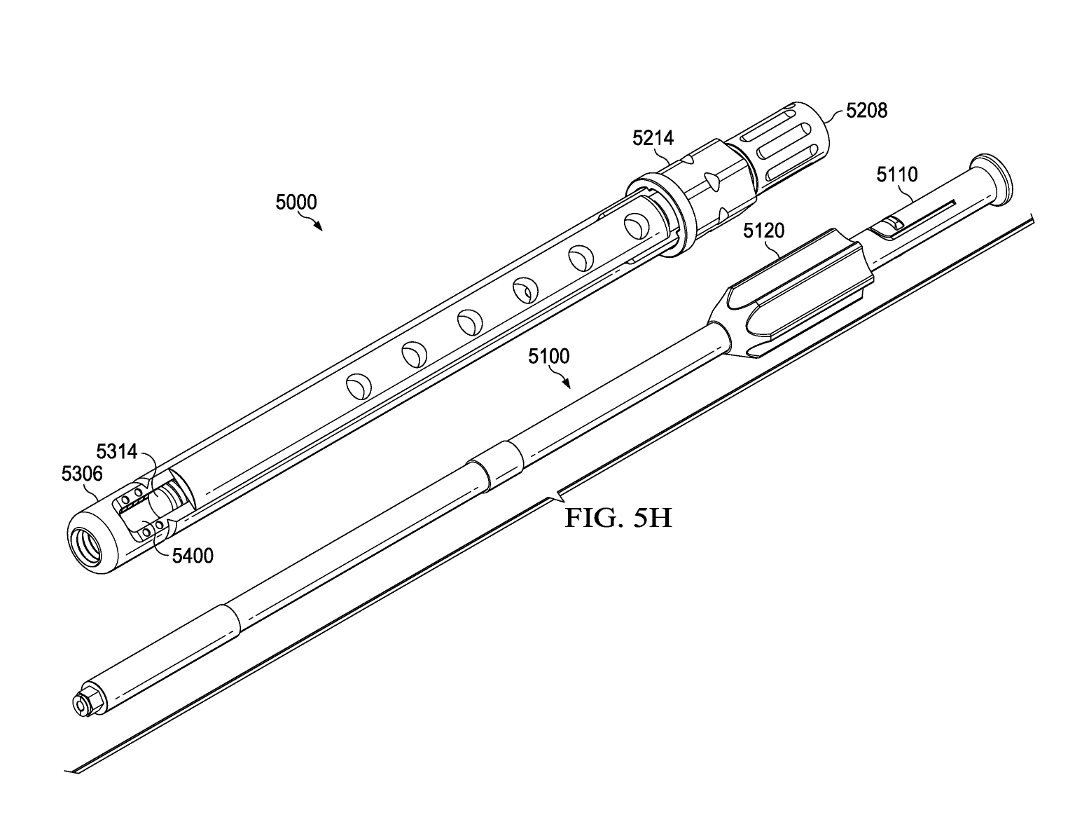

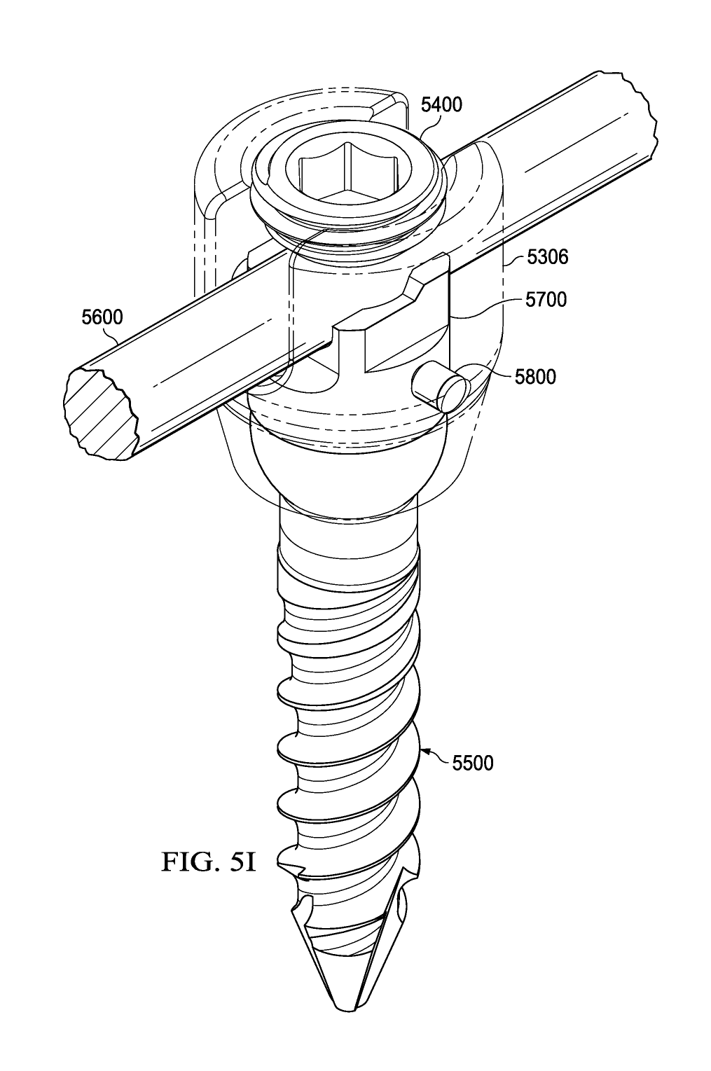

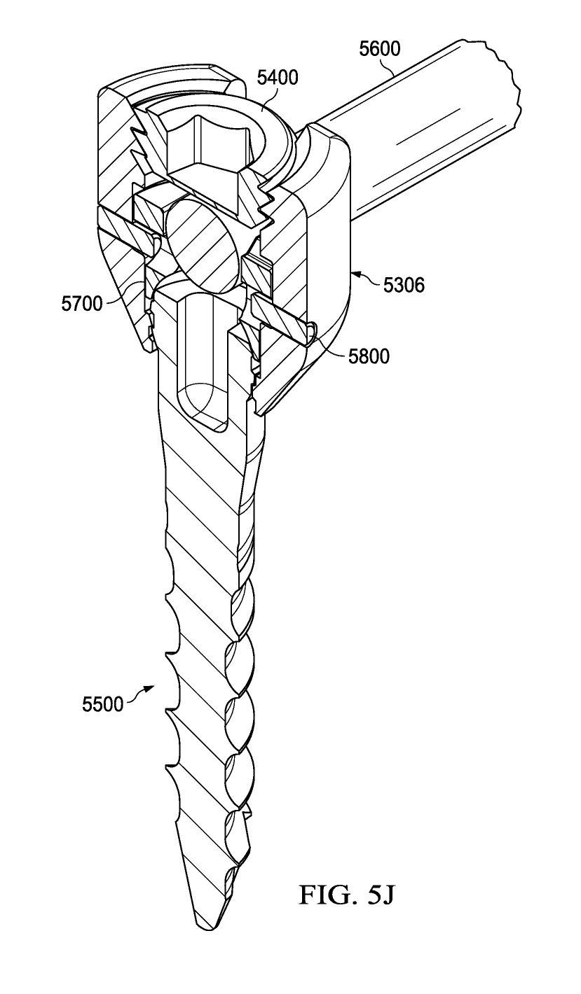

As depicted in FIG. 5A, methods for assembling a spinal fixation system 5000 may further comprise assembling a retention device 5100, such as a set screw holder 5100, by inserting an insert 5110 into the a passageway 5126 of a receiver 5120. As depicted in FIG. 5B, the assembled retention device 5100, such as set screw holder 5100, may be placed through a cannula 5208 of a tower 5200. The set screw holder 5100 may be lowered distally through the cannula 5208. As depicted in FIG. 5C, the set screw holder 5100 may be lowered completely such that a set screw engaging feature 5128 disposed at a distal end 5124 of the receiver 5120 is exposed. The set screw engaging feature 5128 may be exposed between the at least two panels 5210 of the tower 5200. A proximal end 5122 of the receiver 5120 may comprise a larger diameter which may prevent the set screw holder 5100 from being lowered any further into the tower 5200.

Methods may further involve securing a securing device 5400, such as a set screw 5400, to the securing device engaging feature 5128, such as the set screw engaging feature 5128. The set screw engaging feature 5128 may be used in the manner described above in this disclosure. As depicted in FIG. 5D, the set screw 5400 may be secured against the set screw engaging feature 5128 of the set screw holder 5100. The assembled components in FIG. 5D may be in an initial combined arrangement that may be ready for insertion or for securing of the set screw 5400 into a body. As depicted in FIG. 5E, the assembled combination of the set screw holder 5100, the tower 5200 and the set screw 5400 may be lowered through the receiving channel 5314 of the body 5300. The assembled combination may be lowered in such a manner so that the at least two panels 5210 and the at least two tabs 5310 are positioned orthogonally from each other. In such manner, the at least two panels 5210 and the at least two tabs 5310 may not impede or otherwise block the lowering of the assembled combination into the receiving channel 5314. Rather, the orthogonal positioning of the at least two panels 5210 and the at least two tabs 5310 may advantageously provide for improved stability as the assembled combination is disposed into the body 5300. The assembled combination may be lowered until the set screw 5400 comes into contact with the internal threads 5318 of the body 5300. At this point, the assembled combination may not be able to simply be lowered or slid down to further advance the assembled combination.

As previously described (see FIGS. 3C-3E), in some embodiments, a tower 5200 may further comprise an internal shaft having a threading section. In the process depicted in FIG. 5E, methods for certain embodiments may further involve securing a threading section of an internal shaft of the tower 5200 against the internal threads 5318 of the body 5300. Such securing may be accomplished by rotating the tower 5200 so that the threading section mates with the internal threads 5318 to provide for a rigid connection.

Referring to FIG. 5F, methods may further comprise advancing the assembled combination by mating or otherwise engaging the external threads of the set screw 5400 with the internal threads 5318 of the body 5300. Such mating or engaging may involve rotating the set screw holder 5100. The proximal end 5122 of the receiver 5120 of the set screw holder 5100 may be used to provide rotation. Rotating the proximal end 5122 may allow the set screw 5400 secured at the set screw engaging feature 5128 to likewise to rotated and thus be advanced through the internal threads 5318. In some embodiments, such rotation may be accomplished by hand. In some embodiments, such rotation may be accomplished using a separate tool such as a handle adaptor. A handle adaptor may advantageously provide for additional torque which may be desired in certain surgical settings.

As depicted in FIG. 5F, the saddle 5306 at the distal end 5304 of the body 5300 may be empty. However, in some embodiments, various components may already be disposed in the saddle 5306. For example, a bone screw, a mounting rod, a pressure cap, and/or a retention ring may already be disposed in the saddle 5306.

As the set screw 5400 is advanced to the end of the internal threads 5318, a first set of undercuts 5234 of an attachment feature 5214 may receive the at least two tabs 5310 of the body 5300. In some embodiments, a second set of undercuts 5232 of the attachment feature 5214 may receive the at least two panels 5210 of the tower 5200. FIG. 5G depicts a close-up view of the attachment feature 5214, wherein the at least two tabs 5310 are received into a first set of undercuts 5234 of the attachment feature 5214. As depicted FIG. 5G, disposing or securing the at least two tabs 5310 in the attachment feature 5214 may improve stability of the tower 5200 relative to the body 5300.

Referring back to FIG. 5F, the spinal fixation system 5000 as depicted in FIG. 5F may be in a provisionally tightened state. In this state, a handle adaptor, if present, may be removed. Further, in this state, various instruments may be attached to the spinal fixation system 5000. For example, a Firebird Deformity and Phoenix instrumentation for DVR or compression/distraction may be secured on the attachment feature 5214. Such instruments are discussed and provided by way of example only, and one of ordinary skill in the art having the benefit of the present disclosure would appreciate other appropriate instruments for compress/distraction or other surgical maneuvers or manipulation. As another example, compressor/distractor posts may be inserted into any one of the plurality of recesses 5216 of the tower 5200. In some embodiments, it may be advantageous to insert compressor/distractor posts at the recess 5216 closest to a skin surface outside of a surgical incision. Usage of instruments such as the Firebird Deformity and Phoenix instrumentation for DVR or compression/distraction may advantageously allow for positioning of components of the spinal fixation system 5000 to be adjusted, tightened, or better secured. The instruments described here as provided by way of example only, and other appropriate instruments may be used without departing from the present disclosure.

Instruments may be used to allow for rod reduction, direct vertebral rotation, compression, distraction, counter-torque during tightening, and other surgical maneuvers. Rod reduction may comprise exerting a compressive force upon a mounting rod. Direct vertebral rotation may comprise adjusting the orientation of a spinal fixation system or otherwise rotating a vertebral body or bodies so as to change rotational alignment to other vertebral bodies. Compression may comprise moving two or more vertebral bodies, along with their corresponding saddles and bone screws, in closer proximity to one another. Distraction may comprise moving two or more vertebral bodies, along with their corresponding saddles and bone screws, further apart from one another. Counter-torque during tightening may comprise further securing a set screw against the rod within a saddle to provide for additional compressive forces.

Once the desired adjustments and/or tightening have been achieved, the set screw holder 5100 may be disengaged from the set screw 5400. Such action may be accomplished by pulling back the insert 5110 from a locked position. Pulling back the insert 5110 may release the set screw 5400 from the set screw engaging feature 5128 of the set screw holder 5100. As depicted in FIG. 511, the set screw 5400 may remain in a secured against the internal threads 5318 (e.g., shown in FIG. 5E) and may remain within the saddle 5306. In this state, components of the set screw holder 5100, including the insert 5110 and the receiver 5120 may be removed from the assembled spinal fixation system 5000. Said components may be described as a releasable combined arrangement which may be removed from the assembled spinal fixation system 5000.

If further or final tightening is desired, a counter-torque handle may be placed over the attachment feature 5214. For example, an instrument handle of a counter-torque wrench may be fitted over the attachment feature 5214. Further, a set screw driver may be attached to a torque limiting handle. The set screw driver may be inserted down the now unoccupied cannula 5208 to engage the set screw 5400 for further tightening. The set screw 5400 may be tightened against other components in the saddle 5306. As shown in FIG. 5I and FIG. 5J, other components in the saddle 5306 may comprise a bone screw 5500, a mounting rod 5600, a pressure cap 5700, and a holding pin 5800. The set screw driver may allow for tightening of the set screw 5400 against such components to increase stability and security of a bone screw assembly. In some embodiments, the set screw driver may be integrated as part of the spinal fixation system. For example, in some embodiments, a capture mechanism (e.g. small pin engaging a slot on the set screw driver) may be used to integrate the set screw driver to the spinal fixation system. Once the set screw 5400 has been sufficiently tightened or secured in the saddle 5306, the tower 5200 and any other instruments attached thereto may be removed. Methods may then further comprise separating the at least two tabs 5310 (e.g., shown in FIG. 5E and FIG. 5F) from the saddle 5306.

Embodiments of the present disclosure may provide for various advantages during surgical settings and/or for completing various surgical maneuvers. Embodiments of the present disclosure may provide for a smaller overall envelope, and thus a smaller overall incision size. A smaller overall incision size may promote various surgical advantages associated with minimally invasive surgery.

Embodiments of the present disclosure may also provide for great versatility through the previously described features operable to engage with various other instruments for performing surgical maneuvers such as rod reduction, direct vertebral rotation, compression, distraction, and counter-torque during tightening. Incorporating such versatility in a spinal fixation system may advantageously provide for a significant decrease in surgical time. A decrease in surgical time may be advantageous particularly for complex cases that may require various combinations of the aforementioned surgical maneuvers.

Components of the spinal fixation system of the present disclosure may be manufactured or constructed from various appropriate materials. Such materials may be suitable for surgical use. In some embodiments, appropriate materials may include titanium, titanium alloys (ex. Ti-6Al-4V), aluminum, stainless steel, cobalt chrome alloy, polymer (ex. Radel, Ultem, or PEEK) and/or carbon filled polymer. Other appropriate materials may be used without departing from the present disclosure.

As will be understood by one of ordinary skilled in the art who have the benefit of the instant disclosure, other equivalent or alternative compositions, devices, methods, and systems for spinal fixation systems can be envisioned without departing from the description contained herein. Accordingly, the manner of carrying out the disclosure as shown and described is to be construed as illustrative only.

One of ordinary skilled in the art may make various changes in the shape, size, number, and/or arrangement of parts without departing from the scope of the instant disclosure. For example, the position and number of plurality of recesses 3216 of the tower 3200 may be varied. In some embodiments, bodies 1300 and set screws 1400 may be interchangeable. Interchangeability may allow components of a spinal fixation system to be custom adjusted (e.g., by dimensions). In addition, the size of a device and/or system may be scaled up (e.g., to be used for adult subjects) or down (e.g., to be used for juvenile subjects) to suit the needs and/or desires of a practitioner. Each disclosed method and method step may be performed in association with any other disclosed method or method step and in any order according to some embodiments. Where the verb “may” appears, it is intended to convey an optional and/or permissive condition, but its use is not intended to suggest any lack of operability unless otherwise indicated. One of ordinary skill in the art may make various changes in methods of preparing and using a composition, device, and/or system of the disclosure

Also, where ranges have been provided, the disclosed endpoints may be treated as exact and/or approximations as desired or demanded by the particular embodiment. Where the endpoints are approximate, the degree of flexibility may vary in proportion to the order of magnitude of the range. In addition, it may be desirable, in some embodiments, to mix and match range endpoints. In some embodiments, each figure disclosed (e.g., in one or more of the examples, tables, and/or drawings) may form the basis of a range and/or a range endpoint.

All or a portion of a device and/or system for spinal fixation systems may be configured and arranged to be disposable, serviceable, interchangeable, and/or replaceable. These equivalents and alternatives along with obvious changes and modifications are intended to be included within the scope of the present disclosure. Accordingly, the foregoing disclosure is intended to be illustrative, but not limiting, of the scope of the disclosure as illustrated by the appended claims.

The title, abstract, background, and headings are provided in compliance with regulations and/or for the convenience of the reader. They include no admissions as to the scope and content of prior art and no limitations applicable to all disclosed embodiments.

1. A spinal fixation system, comprising:
a body having a proximal end and a distal end, wherein the body comprises:
a saddle at the distal end of the body;
at least two tabs connected to and extending proximally from the saddle;
a receiving channel disposed between the at least two tabs and extending into the saddle; and
a securing mechanism;
a tower having a proximal end and a distal end, wherein the tower comprises:
a central shaft having a proximal end and a distal end, wherein the central shaft is configured to be received into at least a portion of the receiving channel of the body;
a cannula disposed along a longitudinal axis of the central shaft, wherein the longitudinal axis extends from the proximal end to the distal end of the central shaft;
at least two panels disposed at the distal end of the central shaft and extending distally from the central shaft;
an enlarged section disposed at the proximal end of the central shaft; and
an attachment feature disposed at the proximal end of the central shaft, wherein the attachment feature is configured to slide proximally and distally between the at least two panels and the enlarged section;
a retention device, wherein the retention device comprises:
a receiver having a proximal end and a distal end;
wherein the receiver comprises a passageway extending from the proximal end to the distal end of the receiver,
wherein the receiver comprises a securing device engaging feature disposed at the distal end of the receiver, and
wherein the receiver is sized to be received into the cannula of the tower;
an insert having a proximal end and a distal end,
wherein the insert is sized to be received into the passageway of the receiver;
wherein the distal end of the insert is configured to engage the securing device engaging feature;
a securing device having a proximal end and a distal end, wherein the securing device comprises:
a securing feature at the proximal end of the securing device, wherein the securing feature is configured to engage with the securing device engaging feature of the retention device.

2. The spinal fixation system of claim 1, wherein the securing device is a set screw, wherein the retention device is a set screw holder, and wherein the securing device engaging feature is a set screw engaging feature.

3. The spinal fixation system of claim 1, wherein the securing mechanism comprises internal threads disposed along an internal section of a distal end of the at least two tabs and a proximal end of the saddle

4. The spinal fixation system of claim 1, wherein the securing device is a set screw, and wherein the set screw comprises external threads configured to mate with the securing mechanism of the body.

5. The spinal fixation system of claim 1, wherein the enlarged section comprises an annular protrusion relative to the central shaft.

6. The spinal fixation system of claim 1, wherein an exterior of the attachment feature comprises a geometry selected from one of a triangular, rectangular, pentagonal, hexagonal, heptagonal, or octagonal geometry.

7. The spinal fixation system of claim 1, wherein each of the at least two panels each comprise a plurality of recesses disposed on an exterior surface of the at least two panels.

8. The spinal fixation system of claim 7, further comprising a compression tool configured to engage with the plurality of recesses.

9. The spinal fixation system of claim 7, further comprising a distraction tool configured to engage with the plurality of recesses.

10. The spinal fixation system of claim 1, wherein a distal end of the attachment feature further comprises a first set of undercuts configured to receive the at least two tabs of the body, and a second set of undercuts configured to receive the at least two panels of the tower.

11. The spinal fixation system of claim 1, wherein the cannula of the tower is sized such that the securing device cannot pass therethrough.

12. The spinal fixation system of claim 1, wherein a length of the insert of the retention device is greater than a length of the receiver of the retention device.

13. The spinal fixation system of claim 1, wherein the securing device engaging feature is a set screw engaging feature, and wherein the set screw engaging feature is configured to expand or retract in response to contact from the distal end of the insert.

14. The spinal fixation system of claim 1, wherein the insert of the retention device further comprises an exterior tab disposed on the proximal end of the insert, wherein the exterior tab may bias against an internal feature disposed on an interior surface of the receiver.

15. The spinal fixation system of claim 2, wherein the set screw is secured against the set screw engaging feature of the set screw holder when the set screw engaging feature is in an expanded configuration in the securing feature of the set screw.

16. The spinal fixation system of claim 1, wherein the central shaft of the tower further comprises a threading section at the distal end of the central shaft, wherein the threading section comprises exterior threads disposed within the cannula of the tower, wherein the exterior threads are configured to engage with the internal threads of the body.

17. A spinal fixation system, comprising:
a tower having a proximal end and a distal end, wherein the tower comprises:
a central shaft having a proximal end and a distal end,
a cannula disposed along a longitudinal axis of the central shaft, wherein the longitudinal axis extends from the proximal end to the distal end of the central shaft;
at least two panels disposed at the distal end of the central shaft and extending distally from the central shaft;
an enlarged section disposed at the proximal end of the central shaft; and
an attachment feature disposed at the proximal end of the central shaft, wherein the attachment feature is configured to slide proximally and distally between the at least two panels and the enlarged section; and
a retention device, wherein the retention device comprises:
a receiver having a proximal end and a distal end;
wherein the receiver comprises a passageway extending from the proximal end to the distal end of the receiver,
wherein the receiver comprises a securing device engaging feature disposed at the distal end of the receiver, and
wherein the receiver is sized to be received into the cannula of the tower;
an insert having a proximal end and a distal end,
wherein the insert is sized to be received into the passageway of the receiver;
wherein the distal end of the insert is configured to engage the securing device engaging feature.

18. The spinal fixation system of claim 17, wherein the enlarged section comprises an annular protrusion relative to the central shaft.

19. The spinal fixation system of claim 17, wherein an exterior of the attachment feature comprises a geometry selected from one of a triangular, rectangular, pentagonal, hexagonal, heptagonal, or octagonal geometry.

20. The spinal fixation system of claim 17, wherein each of the at least two panels each comprise a plurality of recesses disposed on an exterior surface of the at least two panels.

21. The spinal fixation system of claim 20, further comprising a compression tool configured to engage with the plurality of recesses.

22. The spinal fixation system of claim 20, further comprising a distraction tool configured to engage with the plurality of recesses.

23. The spinal fixation system of claim 17, wherein a length of the insert of the retention device is greater than a length of the receiver of the retention device.

24. The spinal fixation system of claim 17, wherein the securing device engaging feature is configured to expand or retract in response to contact from the distal end of the insert.

25. The spinal fixation system of claim 17, wherein the insert of the retention device further comprises an exterior tab disposed on the proximal end of the insert, wherein the exterior tab may bias against an internal feature disposed on an interior surface of the receiver.

26. The spinal fixation system of claim 17, wherein the system further comprises
a body having a proximal end and a distal end, wherein the body comprises:
a saddle at the distal end of the body;
at least two tabs connected to and extending proximally from the saddle;
a receiving channel disposed between the at least two tabs and extending into the saddle, wherein the receiving channel is sized to receive the central shaft of the tower; and
a securing mechanism; and
a securing device having a proximal end and a distal end, wherein the securing device comprises:
a securing feature at the proximal end of the securing device, wherein the securing feature is configured to mate with the securing device engaging feature of the retention device.

27. The spinal fixation system of claim 26, wherein the securing device is a set screw, wherein the retention device is a set screw holder, and wherein the securing device engaging feature is a set screw engaging feature.

28. The spinal fixation system of claim 26, wherein the securing mechanism comprises internal threads disposed along an internal section of a distal end of the at least two tabs and a proximal end of the saddle, and wherein the set screw comprises external threads configured to mate with the securing mechanism of the body.

29. The spinal fixation system of claim 26, wherein the cannula of the tower is sized such that the securing feature cannot pass therethrough.

30. A method of assembling a spinal fixation system, the method comprising:
disposing a set screw holder within a tower,
wherein the set screw holder comprises:
a receiver having a proximal end and a distal end;
wherein the receiver comprises a passageway extending from the proximal end to the distal end of the receiver, and wherein the receiver comprises a set screw engaging feature disposed at the distal end of the receiver, and
an insert having a proximal end and a distal end,
wherein the insert is disposed in the passageway of the receiver, and wherein the distal end of the insert is configured to engage the set screw engaging feature;
wherein the tower comprises:
a central shaft having a proximal end and a distal end;
a cannula disposed along a longitudinal axis of the central shaft, wherein the longitudinal axis extends from the proximal end to the distal end of the central shaft;
at least two panels disposed at the distal end of the central shaft and extending distally from the central shaft;
an enlarged section disposed at the proximal end of the central shaft; and
an attachment feature disposed at the proximal end of the central shaft, wherein the attachment feature is configured to slide proximally and distally between the at least two panels and the enlarged section;
wherein the set screw holder is received within the cannula of the tower;
securing a set screw to the set screw engaging feature of the set screw holder,
wherein the set screw comprises,
external threads, and
a securing feature at a proximal end of the set screw, wherein the securing feature is configured to engage with the set screw engaging feature of the set screw holder;
providing a body having a proximal end and a distal end, wherein the body comprises:
a saddle at the distal end of the body;
at least two tabs connected to and extending proximally from the saddle;
a receiving channel disposed between the at least two tabs and extending into the saddle; and
a securing mechanism;
inserting an initial combined arrangement of the set screw holder, the tower, and the set screw through the receiving channel of the body; and
securing the initial combined arrangement against the securing mechanism of the body.

31. The method of claim 30, wherein the securing mechanism of the body comprises internal threads disposed along an internal section of a distal end of the at least two tabs and a proximal end of the saddle.

32. The method of claim 31, wherein securing the initial combined arrangement against the securing mechanism of the body comprises rotating the initial combined arrangement, whereby rotating the initial combined arrangement secures the external threads of the set screw against the internal threads of the body.

33. The method of claim 30, the method further comprising
securing an instrument handle against an exterior of the attachment feature, wherein an exterior of the attachment feature comprises a geometry selected from one of a triangular, rectangular, pentagonal, hexagonal, heptagonal, or octagonal geometry.

34. The method of claim 30, the method further comprising
securing an instrument against one of a plurality of recesses disposed on an exterior surface of the at least two panels.

35. The method of claim 30, the method further comprising
receiving the at least two tabs of the body into a set of undercuts of the attachment feature.

36. The method of claim 30, the method further comprising
biasing an exterior tab disposed on the proximal end of the insert against an internal feature disposed on an interior surface of the receiver.

37. The method of claim 30, the method further comprising
positioning the set screw in a secured position such that at least a portion of the set screw is disposed within the saddle of the body; and
releasing the set screw from the set screw holder by retracting the set screw engaging feature from the securing feature of the set screw,
wherein the set screw engaging feature is configured to expand or retract in response to contact from the distal end of the insert.

38. The method of claim 37, the method further comprising
removing a releasable combined arrangement of the set screw holder and the tower through the receiving channel of the body.

39. The method of claim 38, the method further comprising
separating the at least two tabs of the body from the saddle of the body.
